(19) United States
(12) Patent Application Publication
(10) Pub. No.: US 2024/0272042 A1
(43) Pub. Date: Aug. 15, 2024

(54) THIN-FILM-BASED ASSEMBLY

(71) Applicant: VITROTEM B.V., Amsterdam (NL)

(72) Inventor: Pauline Martha Gerardina VAN DEURSEN, Leiden (NL)

(21) Appl. No.: 18/577,438

(22) PCT Filed: Jul. 8, 2022
(86) PCT No.: PCT/NL2022/050400

§ 371 Date: Jan. 8, 2024

(30) Foreign Application Priority Data
Jul. 8, 2021 (NL) 2028670

(51) Int. Cl. G01N 1/28; G01N 23/04; G01N 33/487

(52) U.S. Cl. CPC G01N 1/2813; G01N 33/48707; G01N 23/04; G01N 2223/418

(57) ABSTRACT

A thin-film-based assembly includes a support base and a thin film placed on the support base. The thin film has a thickness of less than 10 nm, such as, for example, a graphene film. One or more spherical nanoparticles are disposed between the thin film and the support base. The spherical nanoparticles functionally constitute spacers between the thin film and the support base.

CROSS-REFERENCE TO RELATED APPLICATION(S)

This is a National Stage Entry into the United States Patent and Trademark Office from International Patent Application No. PCT/NL2022/050400, filed on Jul. 8, 2022, which relies on and claims priority to Netherlands Patent Application No. 2028670, filed on Jul. 8, 2021, the entire contents of which both of which are incorporated herein by reference.

### FIELD OF THE INVENTION

An aspect of the invention relates to a thin-film-based assembly comprising a thin film placed on a support base. The thin-film-based assembly may be, for example, a preparation of graphene liquid cells. Other aspects of the invention relate to use of a thin-film-based assembly, and a method of forming a thin-film-based assembly.

### BACKGROUND ART

Thin films having a thickness of less than 10 nm, such as, for example, graphene films, find use in a wide range of applications. A thin film may be placed on a support base and thus form a thin-film-assembly. The thin film and the support base may jointly define a space, which may encapsulate a substance or have another function. The thin film provides a relatively high degree of transparency, allowing a relatively precise analysis of what is inside this space and what happens inside the space. The support base itself may comprise a thin film. This allows relatively precise analysis by means of radiation traversing the space through the one and the other thin film. The thin films affect this traversing radiation relatively weakly.

A preparation of graphene liquid cells is an example of a thin-film-based assembly as described hereinbefore. Such a preparation may be obtained using a TEM support as a main base, TEM being an acronym for transmission electron microscope. A TEM support is typically in the form of a metal plate with a grid of relatively small apertures. The metal plate may have a thickness of several millimeters. A grid spacing is typically comprised between 0.01 and 0.5 um. Alternatively, a TEM support may comprise a silicon oxide or silicon nitride disk with an aperture or an array of apertures. The TEM support is provided with a bottom graphene film. The TEM support may be coated with a porous thin support film, which supports the bottom graphene film. A liquid containing one or more samples to be analyzed is deposited on the bottom graphene layer that is present on the TEM support.

A top graphene film is placed onto the bottom graphene film that is present on the TEM support. The liquid containing the one or more samples is thereby sandwiched between these graphene films. An excess amount of liquid may be removed by means of, for example, an absorbent sheet. The top graphene film comes into contact with the bottom graphene film. Nanoscopic pockets of liquid form, in which nanoscopic volumes of the liquid containing the one or more samples to be analyzed are tightly encapsulated between the two aforementioned graphene film. These pockets of liquids are the graphene liquid cells, which may form over a substantial area of the TEM support. Typically, several hundred graphene liquid cells may form on the TEM support. The graphene liquid cells may vary in size, typically having a length and width between 0.01 and 5 μm, which are lateral dimensions, and a thickness between 1 and 100 nanometer.

The preparation of graphene liquid cells thus obtained may be used for imaging samples comprised therein by means of a transmission electron microscope. Since the liquid containing the samples to be imaged is tightly encapsulated between the two graphene films, the preparation of graphene liquid cells can be inserted into a vacuum column of the transmission electron microscope. The thinness of the graphene liquid cells allows high resolution imaging of the samples. An electron beam may traverse a graphene liquid cell though an opening in the TEM support. This traversing electron beam will not significantly be affected by the two graphene films, thanks to their extreme thinness.

Patent publication WO2021123458 describes that a thin film liquid cell suitable for transmission electron microscopy at room temperature is fabricated as follows. A thin film floating on a liquid is prepared. A droplet of the liquid with the thin film floating thereon is transferred to a support by means of a loop. The loop carries the droplet and the droplet carries the thin film during this transfer. Sufficient liquid from the droplet on the support is removed to form the thin film liquid cell

### SUMMARY OF THE INVENTION

There is a need for a thin-film-based assembly in which a space between a thin film and a support base can better approximate desired characteristics.

The invention takes the following into consideration. When a thin film is placed on a support base, it may be difficult to obtain a space between these two entities that has certain desired characteristics, or at least approximates these characteristics sufficiently well. This is mainly due to the fact that handling the thin film is delicate and difficult; the thin film being less than 10 nm thick.

For example, in making a preparation of graphene liquid cells a described hereinbefore, the graphene liquid cells may vary in size to a relatively great extent. There may be relatively few graphene liquid cells that have an appropriate size with respect to samples that are to be analyzed. That is, there may be relatively many that are too large or too small, or both. This may also make that there is a relatively low density of graphene liquid cells that have an appropriate size, which may adversely affect an analysis to be made.

An aspect of the invention, which is defined in claim 1, relates to a thin-film-based assembly. The thin-film-based assembly comprises:
a support base; and
a thin film placed on the support base, the thin film having a thickness of less than 10 nm, wherein the thin-film-based assembly comprises:
at least one spherical nanoparticle comprised between the thin film and the support base, the at least one spherical nanoparticle functionally constituting a spacer between the thin film and the support base.

A further aspect of the invention, which is defined in claim 16, relates to use of a thin-film-based assembly as defined hereinbefore.

Yet a further aspect of the invention, which is defined in claim 17, relates to a method of forming a thin-film-based assembly. The method comprises:
laying out spherical nanoparticles on a support base; and
placing a thin film on the support base, the thin film having a thickness of less than 10 nm, the thin film being placed on the support base so that at least one spherical nanoparticle is comprised between the thin film and the support base, whereby the at least one spherical nanoparticle functionally constitute a spacer between the thin film and the support base.

In each of these aspects, the spherical nanoparticles allow obtaining a space between the support base and the thin film placed thereon that can better approximate certain desired characteristics. This is because the spherical nanoparticles constitute spacers that play a role in defining the aforementioned space. The spherical nanoparticles thus provide a form of control over the space that is formed between the support base and the thin film. The control may be exerted through an appropriate size, or size distribution, and an appropriate density, or density distribution, of the spherical nanoparticles. For example, in a preparation of graphene liquid cells, nanoparticles that are added to a liquid containing samples may provide a higher yield of graphene liquid cells that have an appropriate size with respect to the samples encapsulated in these cells. Moreover, the nanoparticles may also contribute to formation of graphene liquid cells, which allows obtaining a relatively high density of these cells in the preparation.

In forming the thin-film based assembly in accordance with the invention, the spherical nanoparticles may freely assemble. In case spherical nanoparticle density is relatively high, this may result into tight packing in certain areas. In these areas, spaces of concave triangular shape may form between the spherical nanoparticles, with dimensions determined by their diameter. Conversely, in case spherical nanoparticle density is relatively low, the spherical nanoparticles are likely to be randomly distributed over the support base. This may result in “tent-like” encapsulations, each “held up” by a single spherical nanoparticle. The height and lateral size of such an encapsulation is again determined by the diameter of the spherical nanoparticle.

Spherical nanoparticles may be produced in bulk and dispended in liquid or kept free, in air. As such, spherical nanoparticles are typically not inherently fixed to the support base, or the thin film, prior to forming the thin-film based assembly in accordance with the invention. In forming the thin-film based assembly, spherical nanoparticles may become fixated within the assembly. Their freedom of movement before becoming fixated allows facile addition of a large array of spherical nanoparticles to the support base. This, in turn, may result in a large number of cells being formed on a macroscopic area.

For the purpose of illustration, some embodiments of the invention are described in detail with reference to accompanying drawings. In this description, additional features will be presented, some of which are defined in the dependent claims, and advantages will be apparent.

### BRIEF DESCRIPTION OF THE DRAWINGS

FIG. 1 is a schematic cross-sectional diagram of a graphene liquid cell comprising spherical nanoparticles that functionally constitute spacers.

FIG. 2 is a top view photograph of a portion of a preparation of graphene liquid cells.

FIG. 3 is a top view photograph taken at a higher magnification of a smaller portion of the preparation of graphene liquid cells.

FIG. 4 is a schematic cross-sectional diagram of a graphene liquid cell supported by a micro-engineered base.

### DESCRIPTION OF SOME EMBODIMENTS

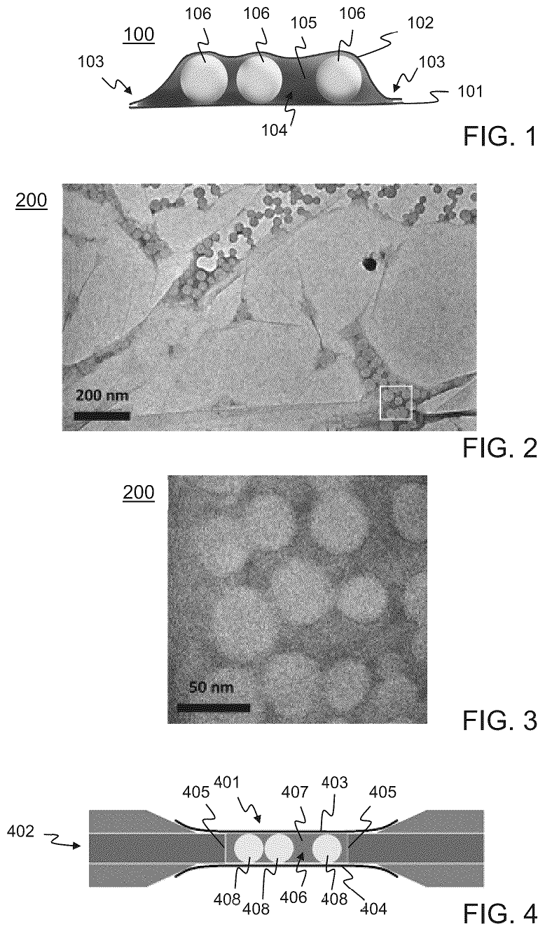

FIG. 1 schematically illustrates a graphene liquid cell 100. FIG. 1 provides a schematic cross-sectional diagram of the graphene liquid cell 100. The graphene liquid cell 100 illustrated in FIG. 1 may form part of a preparation of graphene liquid cells. The preparation of graphene liquid cells may be used for imaging samples comprised therein by means of a transmission electron microscope. The preparation may further comprise a TEM support on which the graphene liquid cell 100 is present, TEM being an acronym for transmission electron microscope. Such a TEM support is not represented in FIG. 1 for the sake of simplicity.

The graphene liquid cell 100 comprises a bottom graphene film 101 and a top graphene film 102. The graphene liquid cell 100 has a circumference 103 that is formed by the top graphene film 102 being locally in contact with the bottom graphene film 101. The top graphene film 102 and the bottom graphene film 101 jointly form a boundary of the graphene liquid cell 100. The top graphene film 102 and the bottom graphene film 101 thus jointly delimit an interior space 104 of the graphene liquid cell 100. The interior space 104 is filled with a liquid 105 containing samples of interest. These samples of interest may include, for example, nanoparticles, biological molecules, or macromolecular assemblies, or any combination of these.

Several spherical nanoparticles 106 are comprised between the top graphene film 102 and the bottom graphene film 101. The spherical nanoparticles 106 are thus present within the interior space 104 of the graphene liquid cell 100. The spherical nanoparticles 106 are arranged in a single layer. The spherical nanoparticles 106 thus functionally constitute spacers between the aforementioned graphene films 101, 102.

The spherical nanoparticles 106 play a main role in defining a thickness of the graphene liquid cell 100, in particular in defining a maximum thickness. The thickness of the graphene liquid cell 100 can thus be adapted to the samples of interest through the use of the spherical nanoparticles 106 having an appropriate diameter. Depending on the samples of interest, the spherical nanoparticles 106 may have a diameter in a range between 1 nanometer and 10 micrometer. More specifically, the spherical nanoparticles 106 may have a diameter less than 3 micrometer, or even less than 1 micrometer. Even more specifically, the spherical nanoparticles 106 may have a diameter less than 100 nm. This then conforms with a definition of the International Organization for Standardization (ISO) according to which a nanoparticle is a discrete nano-object where all three Cartesian dimensions are less than 100 nm. However, spherical nanoparticles that were about 800 nm thick provided satisfactory results in obtaining graphene liquid cells, such as the graphene liquid cell 100 illustrated in FIG. 1. Accordingly, the term nanoparticle may broadly be interpreted in the context of the present application.

The spherical nanoparticles 106 may also play a role in defining lateral dimensions, which includes length and width, of the graphene liquid cell 100. In case the spherical nanoparticles 106 are relatively dense, the top graphene film 102 may span, as it were, several spherical nanoparticles 106 as illustrated in FIG. 1. To that end, the spherical nanoparticles 106 may account for 50% to 90% in at least a portion of the interior space 104 of the graphene liquid cell 100.

Accordingly, the graphene liquid cell 100 illustrated in FIG. 1 may have significantly larger lateral dimensions than a conventional graphene liquid cell, which has been formed without spherical nanoparticles comprised therein. These larger lateral dimensions may leave space for movement, distortion, and aggregation of a sample in the graphene liquid cell 100. This allows imaging such dynamic processes by means of transmission electron microscopy, or other imaging techniques. The lateral dimensions of the graphene liquid cell 100 may be in a range between 10 nanometer and 50 micrometer.

Thus, the spherical nanoparticles 106, which functionally constitute spacers in the graphene liquid cell 100 illustrated in FIG. 1, provide several advantages. First of all, the spherical nanoparticles 106 provide control over the thickness of the graphene liquid cell 100. In addition, the spherical nanoparticles 106 allow the graphene liquid cell 100 to have larger lateral dimensions compared with conventional graphene liquid cells. Finally, it has also been found that the spherical nanoparticles 106 may contribute to a higher yield of suitable graphene liquid cells in a process of making these cells. All in all, the spherical nanoparticles 106 allow control over the graphene liquid cell 100, so that the cell may be better adapted with respect to the samples comprised therein.

For example, let it be assumed that the samples of interest include individual proteins, which have a nanometer size, typically less than 1 nm. For these samples, an appropriate size of the graphene liquid cell 100 is in the order of 100 nm×100 nm×10 nm, whereby 100×100 nm are the lateral dimensions and 10 nm the thickness. As another example, the samples of interest may include vesicles and viruses, which may have a size in the order of tens of nanometer, such as, for example, between 50 and 100 nm. For these samples, an appropriate size of the graphene liquid cell 100 is in the order of 300 nm×300 nm×100 nm, whereby 300 nm×300 nm are the lateral dimensions and 100 nm is the thickness. As yet another example, the samples of interest may include entire biological cells or complex samples that have different parts. For these samples, and appropriate size of the graphene liquid cell 100 is in the order of 1000 nm×1000 nm'500 nm, whereby 1000 nm×1000 nm are the lateral dimensions and 500 nm is the thickness.

FIG. 2 illustrates a preparation of graphene liquid cells 200. FIG. 2 provides a top view photograph of a portion of the preparation of graphene liquid cells 200. This portion comprises several graphene liquid cells, which are relatively large. The graphene liquid cells may have a structure corresponding with that of the graphene liquid cell 100 schematically illustrated in FIG. 1 and described hereinbefore.

FIG. 3 illustrates a smaller portion of the preparation of graphene liquid cells 200 in greater detail. FIG. 3 provides a top view photograph of this smaller portion taken at a higher magnification. A similar smaller portion is indicated in FIG. 2 by means of a white-lined rectangle. The close-up photograph of FIG. 3 shows relatively bright round regions. These correspond with spherical nanoparticles. Darker regions correspond with liquid that is present in between the spherical nanoparticles.

A preparation of graphene liquid cells 200, such as that illustrated in FIGS. 2 and 3, may be obtained in the following manner. It is assumed that a liquid containing samples of interest has already been prepared, which will be referred to as sample-containing liquid hereinafter. It is further assumed that a TEM support has first been coated with a porous thin support film and has then been provided with a bottom graphene film.

Spherical nanoparticles, which may be dry, may be added to the sample-containing liquid and may then be mixed. Accordingly, a dispersion of the spherical nanoparticles in the sample-containing liquid may be obtained, which will be referred to as sample-and-nanoparticle dispersion hereinafter. As explained hereinbefore, the spherical nanoparticles have a diameter that depends on a desired thickness of the graphene liquid cells to be formed. The desired thickness is typically related to the samples of interest. The spherical nanoparticles are added to sample-containing liquid in a quantity so that the spherical nanoparticles in the sample-and-nanoparticle dispersion have an appropriate density. As explained hereinbefore, the appropriate density depends on desired lateral dimensions of the graphene liquid cells to be formed. The desired lateral dimensions are also typically related to the samples of interest.

In an alternative embodiment, a dispersion of spherical nanoparticles in liquid may first separately be prepared. This dispersion may then be added to the sample-containing liquid, which may then be mixed with each other. The appropriate density of the spherical nanoparticles may be obtained taking into account the following factors. A first factor concerns a concentration of the spherical nanoparticles in the dispersion that has been prepared first. A second factor concerns a volume ratio between this dispersion and the sample-containing liquid, which are mixed with each other. This determines an extent to which the concentration of spherical nanoparticles will be diluted in the sample-and-nanoparticle dispersion.

The sample-and-nanoparticle dispersion is deposited on the bottom graphene film that is present on the TEM support. A technique described in patent publication WO2021123458 may be used for that purpose. According to this technique, a droplet of the sample-and-nanoparticle dispersion carries a top graphene film. Accordingly, when the droplet is deposited on the bottom graphene film that is present on the TEM grid, at least a portion of the droplet is sandwiched between the top graphene film and the bottom graphene film. An excess amount of the sample-and-nanoparticle dispersion may then be removed by means of, for example, an absorbent sheet. The top graphene film comes into contact with the top graphene film. Nanoscopic pockets of the sample-and-nanoparticle dispersion form, in which nanoscopic volumes of the sample-and-nanoparticle dispersion are tightly encapsulated between the two aforementioned graphene film. These pockets constitute the graphene liquid cells in which the spherical nanoparticles functionally constitute spacers between the top graphene film and the bottom graphene film as illustrated in FIG. 1 and discussed hereinbefore.

The spherical nanoparticles mentioned hereinbefore may be, for example, spherical nanoparticles developed for quite different purposes, such as, for example, filtering and purifying substances. A spherical nanoparticle has a relatively high surface-area-to-volume ratio: the spherical nanoparticle has a surface area that is relatively large with respect to its volume. Indeed, when a spherical is reduced in diameter, its surface area increases exponentially with respect to its volume. For example, a quantity of spherical nanoparticles of 10 nm in diameter that fill a 6 ml teaspoon has more surface area than a dozen double-sized tennis courts. Their relatively high surface-area-to-volume ratio make that spherical nanoparticles are particularly suited for filtering and purifying substances, and are also suited as active pharmaceutical ingredients. Since the spherical nanoparticles have a relatively large surface area, this allows an extensive interaction with a surrounding substance, which is important for filtering and purifying, as well as a high dissolution rate, which may be desired for active pharmaceutical ingredients.

The spherical nanoparticles may have a main body of inorganic material. The inorganic material may comprise at least one of the following: a polymer, a metal, a metal oxide, a silicate and a ceramic. The polymer may be, for example, polystyrene or polyethylene, or a combination of these. The metal may be, for example, gold or platina, or an alloy.

The spherical nanoparticles may comprise a coating on the main body. The coating may comprise an organic material. The organic material may have an affinity with respect to at least some of the samples comprised in the graphene liquid cell. This affinity may provide a fixating effect on these samples, rather than the samples freely floating in the liquid within the graphene liquid cell. The fixating effect may further prevent a sample from having interaction with a liquid cell wall such as, for example, sticking to graphene. An antibody and a protein are examples of organic material that provide this fixating effect and that may thus be included in the coating on the main body of the spherical nanoparticles.

FIG. 4 schematically illustrates a graphene liquid cell 401 supported by a micro-engineered base 402. FIG. 4 provides a schematic cross-sectional diagram of the graphene liquid cell 401 supported by the micro-engineered base 402. The micro-engineered base 402 may be in the form of, for example, a silicon-based chip that has been micro-engineered by means of at least one of the following techniques: electron beam lithography, photolithography and focused ion beam milling. The micro-engineered base 402 may form part of, for example, a TEM imaging platform, a lab-on-a-chip device, a micro-electromechanical system (MEMS), as well as others types of as well as nanodevices and microdevices.

In this embodiment, a top graphene film 403 is supported by the micro-engineered base 402 at edge portions of this thin film. Similarly, a bottom graphene film 404 is supported by the micro-engineered base 402 at edge portions of this thin film. The two aforementioned graphene films 403, 404 form a top seal and a bottom seal, respectively, of the graphene liquid cell 401, which may be further delimited by side edges 405 of the micro-engineered base 402. The aforementioned entities define an interior space 406 of the graphene liquid cell 401. Like in the graphene liquid cell 100 illustrated in FIG. 1, this interior space 406 may be filled with a liquid 407 containing samples of interest. The side edges 405 may present at least one orifice that allows an inflow of fluid into the interior space 406, or an outflow of fluid, or both.

In this embodiment too, several spherical nanoparticles 408 are comprised between the top graphene film 403 and the bottom graphene film 404. The spherical nanoparticles 408 functionally constitute spacers between the aforementioned graphene films 403, 404. The spherical nanoparticles 408 may also play a main role in defining a thickness of the graphene liquid cell 401 illustrated in FIG. 4. The spherical nanoparticles 408 may further play a main role in defining a form of the graphene liquid cell 401. The spherical nanoparticles 408 thus provide control over the thickness of the graphene liquid cell 401 and may also provide control over the form of the graphene liquid cell 401 that is supported by the micro-engineered base 402.

The embodiments described hereinbefore with reference to the drawings are presented by way of illustration. The invention may be implemented in numerous different ways. In order to illustrate this, some alternatives are briefly indicated.

The invention may be applied in numerous types of products or methods related to thin-film-based assemblies. In the presented embodiments, the thin-film-based assemblies are in the form of graphene liquid cells, in which a thin film comprises graphene. In other embodiments, the thin film may comprise another material, such as, for example other so-called two-dimensional materials such as, for example, hexagonal borin nitride, stacks of several layers of two-dimensional materials, or other thin (up to 10 nm) materials, such as silicon nitride film or amorphous carbon film. The thin film has a thickness of less than 10 nm or, more specifically, less than 5 nm or, even more specifically, less than 2 nm or, yet even more specifically, less than 1 nm. The thin film may be non-porous.

A thin-film-based assembly in accordance with the invention may serve numerous different purposes including, but not limited to, sample analysis and, more specifically, sample analysis by means of a transmission electron microscope. For example, a thin-film-based assembly in accordance with the invention may be used to define a space between two thin film surfaces in which a fluid should flow, which may be a gas or liquid. In embodiments where the thin-film-based assembly comprises cells, these may be other than of graphene liquid cells, which have been discussed by way of illustration.

Although in a thin-film-based assembly at least one nanoparticle functionally constitutes a spacer, this does not exclude embodiments where a sample also functionally constitutes a spacer, jointly with one or more nanoparticles. This specifically applies to embodiment where samples are relatively large, such as, for example, entire biological cells or complex samples that have different parts, as mentioned hereinbefore. For example, a biological cell having lateral dimensions of about 5 micrometer and a height of about 1 micrometer, may be surrounded by at several nanoparticles that are about 1 micrometer thick. Jointly, these may functionally constitute spacers in a graphene liquid cell, or in another form of thin-film-based assembly.

There are numerous different ways of implementing a thin-film-based assembly in accordance with the invention. In the embodiments presented hereinbefore, the thin-film-based assembly comprises a liquid encapsulated by the thin film and the support base, which may comprise a further thin film. In other embodiments, the thin-film-based assembly need not comprise a liquid because, for example, the spherical nanoparticles are employed to facilitate spanning of the thin film over the support base. Such an embodiment allows, for example, measuring conductivity of the thin film without this requiring a substrate that is in contact with the thin-film, which may affect such a measurement. Since the thin film is in contact with the spherical nanoparticles, which functionally constitute spacers, rather than being in contact with the support base, truer measurements can be made. Namely, the spherical nanoparticles may affect the electrical properties of the thin film, as well as other properties, to a significantly lesser extent than the support base would if it were in contact. This example also illustrates that, in a thin-film-based assembly in accordance, the support base need not comprise a further thin film.

There are numerous different ways of forming a thin-film-based assembly in accordance with the invention. As described, a dispersion of spherical nanoparticles in liquid may be added to the support base. As another example, spherical nanoparticles may be sprayed onto the support base. These spherical nanoparticles may be dry. Patterning of surface properties of the support base may assist in obtaining a particular distribution of spherical nanoparticles on the support base.

The term “spherical” used as an adjective for nanoparticle should be interpreted broadly. This term encompasses any shape that allows a nanoparticle to roll, as it were, on the support base before the thin-film-based assembly is definitely formed. For example, the photograph provided in FIG. 3 illustrates that spherical nanoparticles need not be perfectly spherical, or almost perfectly spherical.

The remarks made hereinbefore demonstrate that the embodiments described with reference to the drawings illustrate the invention, rather than limit the invention. The invention can be implemented in numerous alternative ways that are within the scope of the appended claims. All changes that come within the meaning and range of equivalency of the claims are to be embraced within their scope. Any reference sign in a claim should not be construed as limiting the claim. The verb “comprise” in a claim does not exclude the presence of other elements or other steps than those listed in the claim. The same applies to similar verbs such as “include” and “contain”. The mention of an element in singular in a claim pertaining to a product, does not exclude that the product may comprise a plurality of such elements. Likewise, the mention of a step in singular in a claim pertaining to a method does not exclude that the method may comprise a plurality of such steps. The mere fact that respective dependent claims define respective additional features, does not exclude combinations of additional features other than those reflected in the claims.

1. A thin-film-based assembly, comprising:
a support base; and
a thin film placed on the support base, the thin film having a thickness of less than 10 nm,
wherein the thin-film-based assembly comprises
at least one spherical nanoparticle comprised between the thin film and the support base, the at least one spherical nanoparticle functionally constituting a spacer between the thin film and the support base.

2. A thin-film-based assembly according to claim 1, wherein the at least one spherical nanoparticle has a diameter in a range between 1 nanometer and 1 micrometer.

3. A thin-film-based assembly according to claim 1, wherein several spherical nanoparticles account for 50% to 90% in at least a portion of a space between the thin film and the support base.

4. A thin-film-based assembly according to any of claim 1, comprising a cell having a circumference formed by the thin film locally being in contact with the support base, the at least one spherical nanoparticle defining a thickness of the cell.

5. A thin-film-based assembly according to claim 4, wherein the cell has lateral dimensions in a range between 10 nanometer and 50 micrometer.

6. A thin-film-based assembly according to claim 4, wherein the cell comprises a liquid.

7. A thin-film-based assembly according to any of claim 4, wherein the cell comprises a sample to be analyzed.

8. A thin-film-based assembly according to claim 1, wherein the support base comprises a further thin film, the at least one spherical nanoparticle functionally constituting a spacer between the thin film and the further thin film.

9. A thin-film-based assembly according to claim 1, wherein the at least one spherical nanoparticle has a main body of inorganic material.

10. A thin-film-based assembly according to claim 9, wherein the inorganic material comprises at least one of the followings: a polymer, a metal, a metal oxide, a silicate and a ceramic.

11. A thin-film-based assembly according to claim 9, wherein the at least one spherical nanoparticle comprises a coating on the main body.

12. A thin-film-based assembly according to claim 11, wherein the coating comprises an organic material.

13. A thin-film-based assembly according to claim 7, wherein the organic material has an affinity with respect to the sample comprised in the cell, the affinity having a fixating effect on the sample within the cell.

14. A thin-film-based assembly according to claim 13, wherein the organic material comprises at least one of the following: an antibody and a protein.

15. A thin-film-based assembly according to claim 1, wherein the thin film comprises graphene.

16. Use of a thin-film-based assembly according to claim 7 for analyzing the sample that is comprised in the cell.

17. A method of forming a thin-film-based assembly, comprising:
laying out spherical nanoparticles on a support base; and
placing a thin film on the support base, the thin film having a thickness of less than 10 nm, the thin film being placed on the support base so that at least one spherical nanoparticle is comprised between the thin film and the support base, whereby the at least one spherical nanoparticle functionally constitute a spacer between the thin film and the support base.

18. A method of forming a thin-film-based assembly according to claim 17, comprising:
forming a cell by bringing the thin film locally in contact with the support base, at least one of the spherical nanoparticle defining a thickness of the cell.

19. A method of forming a thin-film-based assembly according to claim 17, wherein the at least one spherical nanoparticle is laid out on the support base together with a sample to be analyzed, whereby the spherical nanoparticle and the sample are comprised in a medium.
